(19) United States
(12) Patent Application Publication
(10) Pub. No.: US 2013/0216591 A1
(43) Pub. Date: Aug. 22, 2013

(54) Composition and method for inducing antigen-specific tolerance using IgA

(71) Applicant: Haruyuki Sumimoto, Hirosima city (JP)

(72) Inventor: Haruyuki Sumimoto, Hirosima city (JP)

(21) Appl. No.: 13/740,277

(22) Filed: Jan. 14, 2013

(30) Foreign Application Priority Data
Feb. 19, 2012 (JP) 2012-50983

(51) Int. Cl. A61K 39/395; A61K 39/00; A61K 39/35

(52) U.S. Cl. CPC A61K 39/395; A61K 39/35; A61K 39/0008; A61K 39/001; A61K 39/3955
USPC 424/400; 424/130.1; 424/171.1; 424/158.1; 424/172.1

(57) ABSTRACT

The aim of this invention is to induce and maintain tolerance to specific antigens with IgA (Immunoglobulin A). It is theoretically likely that the major role of IgA is to induce and maintain tolerance because: it binds to epitopes(determinants) on the antigens; it does not mount destruction nor elimination of antigens; almost all antigens in the small intestine are not immunogens but tolerogens; and it is more important to induce tolerance rather than to stimulate immune responses in the small intestine. In other words, IgA is the inhibitor that suppresses IgM-, IgG- and IgE-mediated immune responses. Therefore, In order to induce tolerance to an antigen, it is theoretically effective to administrate the antigen and immunoglobulin A antibodies that are binding to the epitopes of the antigen.

### CROSS-REFERENCE TO RELATED APPLICATIONS

Foreign Application Priority Data

The present application claims priority to Japanese Patent Application JP 2012-50983 filed in the Japan Patent Office on Feb. 19, 2012.

### BACKGROUND

So far, most immunologists have focused on the immune responses, such as destruction of microbes and virus-infected cells, and elimination of parasites.

There are two types of adaptive immunity: humoral immunity and cell-mediated immunity. Humoral immunity is mediated by proteins(immunoglobulin) called antibodies, which are produced by B cells. Cell-mediated immunity is mediated by T cells. CD8+ cytotoxic T lymphocytes kill any cell containing microbes or microbial proteins in the cytoplasm.

There are five classes of immunoglobulin(Ig)—IgM, IgG, IgE, IgD and IgA. Membrane-bound IgM functions as surface receptor of naive B cells. Secreted IgM pentamers are specialized to activate complement reaction efficiently upon binding antigen. IgG also activates complement reaction. The products of the complement reaction kill microbes and coat (opsonize) them for phagocytosis by macrophages and neutrophils. IgE causes inflammation resulting in elimination of antigens such as parasites. The function of IgD is unknown. It may function as membrane-bound receptor of naive B cells. The function of IgA is believed to defense against microbes at mucosal surfaces.

Whereas, less is known about how immune responses are aborted or suppressed. The immune system is often harmful to the host, for example, it causes allergic and autoimmune diseases. Therefore, it is important to elucidate the mechanisms of tolerance.

The desensitization therapy by injection of allergen solution has been the only fundamental therapy for allergic disease, but injection gives pain, a patient must go to hospital regularly for a long time, and it is not very effective.

The oral desensitization therapy (oral administration of allergen) is a potential therapy, but clinical trials have been not very successful.

The factor VIII is being injected to treat hemophilia but the immune system in hemophiliacs recognizes this protein as immunogen and mount immune responses, resulting in destruction of injected factor VIII.

Understanding, controlling and engineering around tolerance is therefore of interest to the industry.

This disclosure concerns a theoretical discovery of IgA(immunoglobulin A) role on tolerance.

### BRIEF SUMMARY

The aim of this invention is to induce and maintain tolerance to specific antigens with IgA (Immunoglobulin A). It is theoretically likely that the major role of IgA is to induce and maintain tolerance because: it binds to epitopes(determinants) on the antigens; it does not mount destruction nor elimination of antigens; almost all antigens in the small intestine are not immunogens but tolerogens; and it is more important to induce tolerance rather than to stimulate immune responses in the small intestine. In other words, IgA is the inhibitor that suppresses IgM-, IgG- and IgE-mediated immune responses. Therefore, In order to induce tolerance to an antigen, it is theoretically effective to administrate the antigen and immunoglobulin A antibodies that are binding to the epitopes of the antigen.

### DETAILED DESCRIPTION

So far, most immunologists have focused on the immune responses, such as destruction and elimination of pathogens. Whereas less is known about how the immune system recognize food antigens as tolerogen (antigens that induce tolerance), and why some people suffer allergic diseases, hypersensitive to commonly encountered environmental antigens such as house dust and pollen.

Because the structure of monomeric IgA antibodies is almost the same as those of other Ig antibodies, it is believed that the function of IgA must be the same as those of other Ig antibodies. In fact, it is demonstrated that IgA plays a key role in shaping the repertoire of gut microbiota. This means that IgA controls or kills some type of microbes in the gut.

However, from another point of view, the major function of IgA must be to induce and maintain tolerance to antigens.

When I was learning biology, I found out that:

(a) gene expression is often regulated not by activation but by suppression and
(b) there are several organs the main function of which is to suppress inner cells.

I describe two examples on (a): ectoderm differentiation and flower-bud formation.

Cells on the ventral side of the blastula secrete a variety of proteins such as bone morphogenetic protein-4 (BMP-4). This protein prevents the ectodermal cells from following their default pathway to forming neural organizations, resulting in forming epidermis. The organizer secretes chordin and noggin. These proteins bind to BMP-4 in the extracellular space and prevent BMP-4 from binding to receptors on the surface of the overlying ectoderm cells. As a result, the ectodermal cells follow their intrinsic path to forming neural organizations.

A FLC (Flowering Locus C) gene encodes a transcription factor that blocks the expression of the genes needed for flowering. The level of FLC mRNA is high in the fall. Low temperatures cause the suppression of FLC gene in winter. As a result, flower-bud formation begins in spring.

These examples illustrate that gene expression is regulated by suppression. Presumably, a gene will be expressed automatically if there are no suppression.

I describe two examples on (b): the ovary and the thymus.

In female mammals, primary oocytes in the ovary are formed very early before birth and remain arrested in prophase of meiotic division I until the female becomes sexually mature. At this point, a small number periodically mature under the influence of hormones. Most germ cells in the ovary are primary oocytes, and in order to keep them arrested in prophase of meiotic division I, the follicular cells secrete RNA synthesis inhibitor. The function of ovary in humans is to discharges an ovum once a month. But the major function of the ovary may be to suppress primary oocyte's development for long periods.

Most cells developing in the thymus undergo apoptosis during the process of positive and negative selections. Immature T cells that do not recognize an MHC molecule in the thymus die by apoptosis in the positive selection. Immature T cells whose receptors strongly recognize MHC-peptide complexes in the thymus undergo apoptosis in the negative selection. More than 95 percent of immature T cells in the thymus are killed during these selections. The function of thymus is believed to supply harmless T cells to the circulation and the peripheral lymphoid organs. But the major function of thymus may be to suppress or kill potentially harmful T cells.

These examples illustrate that the major function of organs having potentially reactive cells may be to suppress the cells reactivity.

Similarly, the major function of the immune system also may be to suppress the potentially harmful responses. In some cases, the immune system is harmful to the host. It causes hay fever, food allergies, bronchial asthma, anaphylaxis, and autoimmune diseases.

In other words, the immune system is not doing nothing in a normal resting state but suppressing immune responses. And only when it is stimulated and the suppression is broken, it may mount immune responses.

There are two classes of tolerances: tolerance to self antigens and tolerance to foreign antigens.

To self antigens, the immune system is unresponsive in normal individuals. Self-reactive T cells are eliminated in the thymus and regulatory T cells suppress other self-reactive cells.

To foreign antigens, especially to food antigens, what are the factors that determine whether the immune system recognize them as immunogens or tolerogens?

If chicken egg albumin is administrated to mice under certain conditions, IgG or IgE antibody is produced. However, if the same albumin is orally administrated previously, antibody is not produced. This phenomenon is called “oral tolerance”.

A likely mechanism is that antigen presenting cells recognize IgA-binding antigens as tolerogens, because mucus in the small intestine contains abundant IgA and food antigens are absorbed with IgA. In other words, IgA may be tolerance marker of the food antigen.

Another likely mechanism is that antigen presenting cells that are resident in the small intestine do not express immune response-triggering costimulator, and they express immune-suppressing costimulator.

It is known that antigen presenting cells that are resident in peripheral lymphoid and nonlymphoid tissues are normally in a resting state and express few or no costimulators as described on page 224 chapter 10 in “Cellar and Molecular Immunology 5th edition updated edition” edited by Abul K. Abbas et al published by P A Saunders in 2005 (ISBN:1416023895), the teachings of which are hereby incorporated by reference.

The detailed reasons why IgA must be tolerance inducer are as follows.

Firstly, the amount of IgA produced is the largest of all immunoglobulins.

The amount of immunoglobulins that a person weighing 60 kg synthesizes in a day are as follows:

| | |
|---|---|
| IgA | 4 g |
| IgG | 0.034 g |
| IgM | 0.008 g |
| IgE | 0.00002 g |

These values illustrates that the major work of whole B cells is to synthesize IgA. If the major work of whole immune system is to induce and maintain tolerance and a majority of the immunoglobulins produced are IgA, it is likely that IgA contribute to tolerance.

Secondly, IgA is the principal antibody isotype that is secreted mainly in mucus.

There are three principal route through which foreign antigen enter the host:
food antigens enter the host through the epithelia of the small intestine;
floating dust(e.g. house dust and pollen) antigens enter the host through the epithelia of the respiratory tracts; and
other antigens enter the host through the epithelia of the skin.

In most cases, food antigens are harmless to the host. It is likely that there is something binding to food antigens which indicate that the antigen is tolerogen. IgA is abundant in mucus such as intestinal mucosa, saliva and mother's breast milk.

Thirdly, IgA does not cause destruction nor elimination of antigens. IgM, IgG and IgE cause complement reactions or inflammation, resulting in destruction or elimination of antigens. However, IgA has no such functions as described on page 412 chapter 17 in “Immunology, infection, and immunity” edited by Gerald B. Pier et al published by ASM Press in 2004 (ISBN:1555812465), the teachings of which are hereby incorporated by reference. IgA only attaches to the epitopes on the antigen. Epitope is the specific portion of a macromolecular antigen to which an antibody binds.

Fourth, T cell activation requires recognition of MHC-associated peptide antigen presented by antigen presenting cells. And a antigen presenting cell can not recognize a specific antigen as tolerogen without additional informations. It is likely that IgA-binding may indicate that the antigen is tolerogen.

Fifth. The antigen receptors of naive B cells, which are mature B cells that have not encountered antigen, are membrane-bound IgM and IgD. After naive B cells recognize antigens with costimulation, class switching occurs and they differentiate into Ig secreting B cells. If naive B cells recognize antigens without costimulation, they die by the process of apoptosis. When recognize antigens with costimulation inducing immune response, it is known that they differentiate into IgM, IgG or IgE secreting B cells. When recognize antigens with costimulation inducing tolerance, they must differentiate into IgA secreting B cells because IgA does not trigger immune responses.

Sixth, there are the observation that some IgA-deficient individuals have increased uptake of food antigens, circulating food-immunoglobulin complexes, and an increased susceptibility to atopic allergies as described on page 412 chapter 17 in “Immunology, infection, and immunity”, the teachings of which are hereby incorporated by reference. This means that IgA involves in tolerance and allergy in some cases.

Seventh, it is known that TGF-β(transforming growth factor-β) induces B cell switching to IgA and inhibits lymphocyte proliferation as described on page 237 chapter 10 in “Cellar and Molecular Immunology 5th edition updated edition”, the teachings of which are hereby incorporated by reference. This suggests that IgA is involved in tolerance.

For all of these reasons, it is theoretically likely that the major function of IgA is to induce and maintain tolerance to antigens.

So far, there have been no books nor web sites introducing that IgA involves in tolerance. It will be understood that the present discovery was very difficult.

Soon after I presented this theory as IgA tolerance hypothesis on my web site in Japanese language on Feb. 7, 2012, and wrote it to professor Suko, the chief professor of healthcare center of Tokyo University of Arts, he suggested me another possible IgA mechanism on tolerance in his official paper mail.

His suggestion is as follows. After IgA bind to the epitopes of antigens, IgM, IgG and IgE can not bind to the same epitopes, resulting in unresponsiveness to the antigen. This means that IgA functions as competitive inhibitor.

Because induction and maintenance of tolerance is important for homeostasis, there must be multiple mechanisms that contribute to tolerance.

The advantages of competitive inhibition of IgA may be:
to be able to induce tolerance immediately; and
to be able to function even if the receptors or costimulators of antigen presenting cells are deficient.

Whereas the advantages of the mechanism which IgA contribute to as tolerance marker may be:
to be able to enhance suppression of immune responses by T cell proliferation; and
to be able to maintain tolerance for long period by memory cells.

Theoretically, there are at least two pathways that induce tolerance to food antigen. One is the IgA-mediated pathway and another is the pathway initiated by antigen presenting cells in the small intestine.

All the antigen presenting cells may recognize IgA-binding antigens as tolerogen, resulting in inducing tolerance to the antigen. The antigen-specific IgA is provided by the host's memory B cells, or via mother's colostral and breast milk.

In the absence of antigen-specific IgA, antigen presenting cells that are resident in the small intestine may play a key role. They may be different from those that are resident in other cites and may generate signals, such as TGF-β that inhibits B cell proliferation and leads B cells to differentiate into IgA secreting cells.

Little is known about the reason why some individuals suffer allergic diseases, hypersensitive to commonly encountered antigens such as house dust, ragweed or cedar pollen. IgA may be involved in hypersensitivity.
Allergic individuals produce large amounts of IgE antibody in response to commonly encountered antigens, whereas normal individuals do not.
Presumably, the immune system in normal individuals do not ignore commonly encountered antigens but recognize them as tolerogens and produce IgA, resulting in unresponsiveness.

Likely explanations for why babies take milk only and why children select foods carefully and reject some foods in tears are that babies can not produce IgA themselves and children can produce few kinds of IgA respectively. Normal adults can take any food presumably because they have abundant kinds of IgA.

Pediatricians know that babies nourished with breast milk are less susceptible to infections than babies nourished with powdered milk. The difference between breast milk and powdered milk may be whether they contain antibody or not. Babies nourished with powdered milk may become susceptible to infections and hypersensitive to environmental antigens, because powdered milk lack antibody information.

This IgA theory may be confirmed by the experiment as follows. If an antigen is injected into mice under the proper conditions, IgG antibody against the antigen is produced. Therefore, in order to confirm IgA functions, inject the same antigen with IgA under the same condition. The IgA, of course, must be specific to the antigen and binds directly to the epitopes on the antigen.

Because food antigen is degraded into polypeptide fragments by enzymes and the epithelium of the gut open its pore to a radius of 5 nanometers allowing amino acids and polypeptides of up to 11 amino acids to pass through, the epitopes are preferably on the degraded polypeptide fragments.

One of the reasons why the clinical trial of oral tolerance that was performed in University of Tokyo during the 1990's failed was that some patients got diarrhea. The antigen used was house dust and the main antigen protein is the protein of dead body and dung of tick. Diarrhea suggests that memory B cells which secrete IgE specific to the tick antigen are dominant in the allergic patient, and the IgE stimulated inflammation in the intestine.

By the way, knockout mice lacking IgA is not very useful to demonstrate that IgA is involved in tolerance. As described previously, there must be a plurality of mechanisms to induce tolerance because tolerance to food antigens is important to the host and failure of tolerance responses may be life-threatening. Other tolerance mechanisms must work and compensate for the absence of IgA.

Composition Embodiments

A composition for inducing antigen-specific tolerance comprising an antigen and immunoglobulin A antibodies that are binding to the epitopes of the antigen. This means that the IgA is specific to the epitopes. Because epitopes of the antigen are blocked by IgA antibodies, IgE or IgG antibodies can not bind to the epitopes, resulting in suppression of IgE- or IgG-mediated immune responses and leading to antigen-specific tolerance.

So far, administrated composition have never included IgA. The epitopes on the antigen in the administrated composition have bee free to interact with any Ig antibodies. When IgG or IgE antibodies binds to the epitopes, the antigen is recognized by the immune system as immunogen. When naive B cells recognize the antigen and immunogenic costimulation, the B cells differentiate into effector B cells and memory B cells which secrete IgG or IgE specific to the antigen.

Whereas, the composition of the present invention contain not only antigen but also IgA. And the most important point of the present invention is that the IgA is specific to the epitopes of the antigen and binds to the epitopes. The composition of the present invention has never existed in this world. The effect of the present invention is to be capable of suppression of IgE- or IgG-mediated immune responses and leading to antigen-specific tolerance more effectively.

The process of producing IgA antibody specific to an antigen can be conducted using hybridomas. Hybridoma is a cell line derived by cell fusion, or somatic cell hybridization, between a normal lymphocyte and an immortalized lymphocyte tumor line.

Food protein are degraded by enzymes into polypeptide fragments and finally into amino acids in the intestine. And as described previously, polypeptide fragments of up to 11 amino acids are absorbed into body and recognized by the immune system.

Therefore, the degraded polypeptide rather than original protein may be effective to induce tolerance in a case where the antigen is protein. And because the structure of polypeptide is more simple than that of protein, target epitope may be easy to be defined when using the degraded polypeptide rather than when using the original protein.

However, the polypeptide and IgA may be degraded by gastric acid before it reach the small intestine when administrated orally. In order to prevent this, the polypeptide and IgA of the present invention should be enteric-coated.

Most enteric coatings work by presenting a surface that is stable at the highly acidic pH found in the stomach, but breaks down rapidly at a less acidic (relatively more basic) pH. For example, they will not dissolve in the gastric acid of the stomach (pH 2-3), but they will in the alkaline (pH 7-9) environment present in the small intestine.

Moreover, the IgA should be from the same species to which the IgA is administrated. For example, in a case where it is administrated to human, the IgA should be humanized or fully human IgA. Because the human immune system will recognize antibodies from non-human species as immunogen, and will mount a response against them, humanized antibodies are preferable and human IgA is the best. Humanized antibodies are antibodies from non-human species whose protein sequences have been modified to increase their similarity to antibody variants produced naturally in humans. They reduce the human immune responses against them. Human antibodies carry the lowest immunological risk.

As target antigens, there are allergens such as house dust, tick, pollen, and many kinds of food proteins, but allergens are not only target antigens. Other proteins such as powdered milk, factor VIII for hemophiliac and proteins associated with graft cells may be target antigens of the present invention. Any antigen to which tolerance should be induced can be target antigen of the present invention.

Method Embodiments

In one embodiment of the present invention, an antigen and immunoglobulin A antibodies that are binding to the epitopes of the antigen are administrated.

In one embodiment of the present invention, polypeptide and immunoglobulin A antibodies that are binding to the epitopes of the polypeptide are administrated.

In one embodiment of the present invention, polypeptide and immunoglobulin A antibodies that are binding to the epitopes of the polypeptide are enteric-coated and then administrated orally.

The invention may be embodied in other specific forms without departing from the spirit or essential characteristics thereof. The present embodiments are therefore to be considered in all respects as illustrative and not restrictive, the scope of the invention being indicated by the appended claims rather than by the foregoing description and all changes which come within the meaning and range of equivalency of the claims are therefore intended to be embraced therein.

What is claimed is:

1. A composition for inducing antigen-specific tolerance comprising:
an antigen and
immunoglobulin A antibodies that are binding to the epitopes of the antigen.

2. The composition of claim 1 wherein the antigen is polypeptide.

3. The composition of claim 2 wherein the polypeptide and IgA are enteric-coated.

4. The composition of claim 1 wherein the antigen is allergen.

5. The composition of claim 1 wherein the antigen is powdered milk.

6. The composition of claim 1 wherein the antigen is factor VIII for hemophiliac.

7. The composition of claim 1 wherein the antigen is associated with graft cells.

8. A method for inducing antigen-specific tolerance comprising:
administrating an antigen and immunoglobulin A antibodies that are binding to the epitopes of the antigen.

9. The method of claim 8 wherein the antigen is polypeptide.

10. The method of claim 9 wherein the polypeptide and IgA are enteric-coated.
